(19) United States
(12) Patent Application Publication
(10) Pub. No.: US 2019/0211380 A1
(43) Pub. Date: Jul. 11, 2019

(54) BIOMARKER DETECTION

(71) Applicant: SLIVE, INC., Palo Alto, CA (US)

(72) Inventor: Erez PODOLY, Menlo Park, CA (US)

(21) Appl. No.: 16/311,946

(22) PCT Filed: Jun. 20, 2017
(86) PCT No.: PCT/US2017/038316

§ 371 Date: Dec. 20, 2018

Related U.S. Application Data
(60) Provisional application No. 62/352,427, filed on Jun. 20, 2016.

(51) Int. Cl. C12Q 1/6825

(52) U.S. Cl. CPC C12Q 1/6825

(57) ABSTRACT

Provided are methods for detecting biomarkers in an analyte. In some embodiments, the detection is accomplished using a competition reaction between a primer and a biomarker for binding to a salimer. The salimer, in some aspects of the invention, is hybridized to the primer, but because the salimer has a greater affinity for a biomarker than for the primer, the salimer dissociates from the primer in the presence of the target biomarker. The unbound primer generates a signal that indicates the presence of the biomarker.

### CROSS-REFERENCE TO RELATED APPLICATIONS

This application claims the benefit of U.S. Patent Application No. 62/352,427, filed Jun. 20, 2016, the entire contents of which are hereby incorporated by reference herein in their entirety.

### FIELD OF THE INVENTION

This disclosure relates to the field of biomarker detection.

### BACKGROUND OF THE INVENTION

The detection of biomarkers is an important means of surveilling disease status and response to treatment in a patient. Individuals predisposed to disease can monitor the presence or absence of biomarkers as indicators of their health status, and the early detection of changes in their biomarker profile can greatly improve their long-term prognosis as treatment of early stage disease is the most likely to be effective. Present technologies, however, are inconvenient or too expensive for daily monitoring. For example, enzyme linked immunosorbent assay (ELISA), gel electrophoresis, and fluorescence detection methods of determining the biomarker status of a patient require expensive equipment, and the assays are typically performed in a laboratory setting by professional technicians. Thus, there exists a need for an assay independent of expensive machinery and capable of being performed in an at-home setting for daily monitoring of an individual's biomarker profile.

### SUMMARY OF THE INVENTION

Disclosed herein are methods of determining the presence or absence of a target biomarker in an analyte comprising hybridizing a salimer to a primer disposed on a surface of an electrode, the electrode capable of being electrically energized, at a temperature suitable to form a double-stranded hybrid between the salimer and the primer, which allows for detecting a first electrical signal from the electrode with the double-stranded hybrid disposed on the surface of the electrode. Exposing the analyte to the double-stranded hybrid initiates a competition reaction between the primer and the target biomarker for complexing with the salimer, wherein the salimer dissociates from the primer in the presence of the target biomarker and forms a complex with the target biomarker. Detecting a second electrical signal from the electrode and comparing the electrical signals allows for ascertaining the presence or absence of the target biomarker.

Methods are also provided for determining the presence or absence of a biomarker comprising in a reaction chamber, hybridizing at least one single-stranded salimer to at least one single-stranded primer to form at least one double-stranded hybrid, wherein the reaction chamber comprises an interior, an exterior, an inlet connecting the exterior of the chamber and the interior of the chamber, and at least one electrode capable of being electrically energized, wherein the at least one primer is disposed on a surface of the at least one electrode, and wherein the at least one salimer has a greater affinity for a target biomarker than for the at least one primer; detecting a first electrical signal from the electrode with the double-stranded hybrid disposed on the surface of the electrode; delivering an analyte to the interior of the chamber, wherein in the presence of the target biomarker the at least one salimer preferentially interacts with the target biomarker and dissociates from the at least one primer to form a salimer-biomarker complex; detecting a second electrical signal from the at least one electrode; and comparing the first electrical signal to the second electrical signal, a different second signal indicates the presence of a biomarker.

Methods are also provided for detecting the presence or absence of a biomarker in an analyte comprising hybridizing a fluorophore-labeled primer to a dark quencher-labeled salimer to form a double-stranded primer-salimer hybrid; detecting a first fluorescence signal from; exposing the analyte to the double-stranded hybrid to initiate a competition reaction between the primer and the target biomarker for complexing with the salimer, wherein the salimer dissociates from the primer in the presence of the target biomarker and forms a complex with the target biomarker; detecting a second fluorescent signal; and comparing the fluorescent signals to ascertain the presence or absence of the target biomarker.

A method is also provide for identifying a salimer-biomarker binding sequence comprising contacting a salimer-biomarker complex with at least one nuclease under conditions amenable for oligonucleotide digestion; isolating the salimer-biomarker complex from the nuclease; dissociating the salimer from the biomarker; and identifying the salimer-biomarker binding sequence.

Also disclosed herein is a salimer having a higher affinity for a biomarker present in an analyte than for a complimentary oligonucleotide sequence, wherein the biomarker is associated with at least one health condition and/or physiological parameter and/or physiological response to a change in one or more parameters.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods however, the methods is not limited to the specific embodiments disclosed. In the drawings:

FIG. 1 illustrates a working embodiment of the present invention.

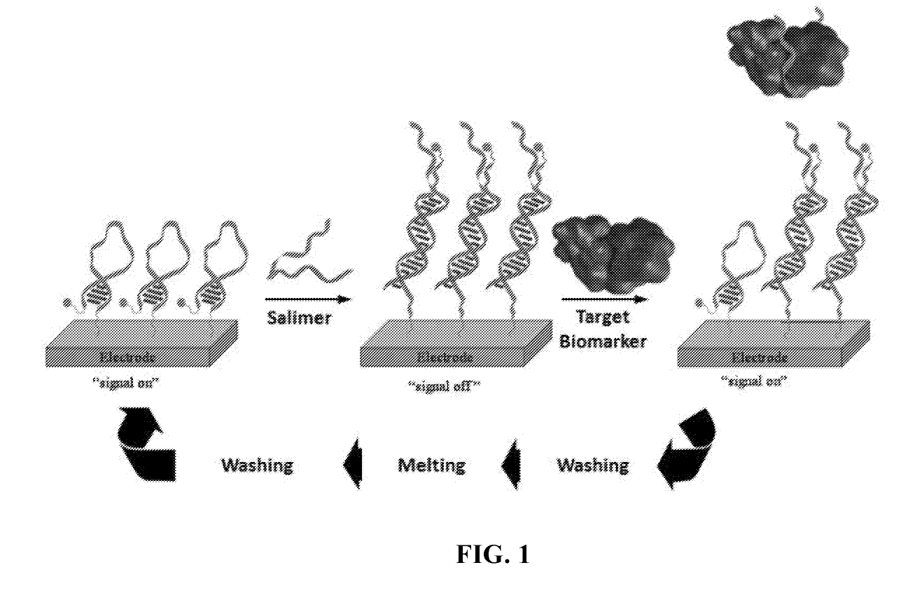

### DETAILED DESCRIPTION OF ILLUSTRATIVE EMBODIMENTS

The disclosed methods may be understood more readily by reference to the following detailed description taken in connection with the accompanying FIGURES, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub-combination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

Biomarker detection is at the epicenter of personalized medicine. Physicians and patients armed with the knowledge of a patient's predisposition to disease or illness can design treatment plans or behavioral adjustments to prevent or delay the onset of symptoms. For those patients who display clinical symptoms of a disease or illness, biomarkers provide a window into the therapies available, the effectiveness of therapy, and the onset of resistance. Because a patient's prognosis is generally better if an abnormality is detected early, biomarker detection is ideal for monitoring one's health status as it allows for early detection and resulting early treatment of abnormalities. Current protocols for biomarker detection are only feasible in a laboratory setting, making this potentially life-saving methodology expensive and inconvenient. The costs associated with laboratory equipment, space, and personnel can make biomarker detection financially impractical for some and unattainable for others.

Methodologies for detecting biomarkers independent of expensive laboratory machinery are needed. Detecting biomarkers on a daily basis in a home or point-of-care setting would increase patient compliance with monitoring their health. Importantly, daily monitoring would allow a patient and their physician to be aware of gradual changes that if addressed early can significantly improve the patient's health.

As described herein, the present invention allows for the detection of a biomarker in a point-of-care or in-home setting, although the invention can be used in laboratories, in the field, or in any other setting where rapid detection of at least one biomarker is desired. One embodiment of the present invention provides a method of determining the presence or absence of a target biomarker in an analyte comprising hybridizing a salimer to a primer disposed on a surface of an electrode, the electrode capable of being electrically energized, at a temperature suitable to form a double-stranded hybrid; detecting a first electrical signal from the electrode with the double-stranded hybrid disposed on the surface of the electrode. Exposing the analyte to the double-stranded hybrid initiates a competition reaction between the primer and the target biomarker for complexing with the salimer, wherein the salimer dissociates from the primer in the presence of the target biomarker and forms a complex with the target biomarker; detecting a second electrical signal from the electrode, the electrical signals are compared to ascertain the presence or absence of the target biomarker.

In some embodiments of the present invention, the hybridization of the primer to the salimer and the competition reaction may occur in a reaction chamber. Thus, the present invention provides a method of determining the presence or absence of a biomarker comprising in a reaction chamber, hybridizing at least one single-stranded salimer to at least one single-stranded primer to form at least one double-stranded hybrid, wherein the reaction chamber comprises an interior, an exterior, an inlet connecting the exterior of the chamber and the interior of the chamber, and at least one electrode capable of being electrically energized, wherein the at least one primer is on a surface of the at least one electrode, and wherein the at least one salimer has a greater affinity for a target biomarker than for the at least one primer; detecting a first electrical signal from the electrode with the double-stranded hybrid disposed on the surface of the electrode; delivering an analyte to the interior of the chamber, wherein in the presence of the target biomarker the at least one salimer preferentially interacts with the target biomarker and dissociates from the at least one primer to form a salimer-biomarker complex; detecting a second electrical signal from the at least one electrode; and comparing the first electrical signal to the second electrical signal, wherein a difference between the first signal and the second signal indicates the presence of a biomarker.

As used herein, “primer” refers to a single-stranded oligonucleotide comprised of a deoxyribonucleotide, a ribonucleotide, a peptide nucleotide, a morpholino, a locked nucleotide, a glycol nucleotide, a threose nucleotide, nucleotides phosphoramidite, any synthetic nucleotides, or any isoforms, combinations, or derivatives thereof. Those skilled in the art will recognize that chemical modification of naturally or non-naturally occurring nucleotides can be used to produce the primers or salimers of the present invention. “Single-stranded” refers to an oligonucleotide not bound by a complimentary strand. Single-stranded oligonucleotides may have internal sequences that are complimentary, which can cause the primer to form secondary structures in some environments. In some aspects, modified, or non-naturally occurring, nucleotides such as those described above can confer altered affinity to the biomarker or the primer. Thus, design of the primer may encompass not just determination of a proper sequence, but also an affinity analysis.

In some aspects of the present invention, the primer of the present invention comprises between about 5 to about 50 nucleotides in length. In other aspects, the primer comprises about 60, 70, 80, 90, or even 100 nucleotides in length. Thus, in some embodiments the primer is between about 10-20 nucleotides in length, between about 20-50 nucleotides in length, or even between about 50-100 nucleotides in length.

A “salimer” is a single-stranded oligonucleotide that comprises deoxyribonucleotide, a ribonucleotide, a peptide nucleotide, a morpholino, a locked nucleotide, a glycol nucleotide, a threose nucleotide, nucleotides phosphoramidite, any synthetic nucleotides, or any isoforms, combinations, or derivatives thereof. Those skilled in the art will recognize that chemical modification of naturally occurring nucleotides or other non-naturally occurring nucleotides, can be used to produce the salimers of the present invention. “Affinity” as used herein refers to the rate at which two or more molecules or compounds bind, hybridize, or otherwise interact. Affinities can be quantified and the affinity constant, Ka, is the product of the binding rate divided by the dissociation rate. Thus, the higher the affinity constant for two compounds, the more likely that the compounds when in proximity will bind, hybridize, or otherwise interact. Some embodiments of the present invention provide a salimer having a higher affinity for a biomarker present in an analyte than for the primer sequence, wherein the biomarker is associated with at least one health condition and/or physiological parameter and/or physiological response to a change in one or more parameters.

Designing primers and salimers that meet the criteria outlined above involves first determining oligonucleotide sequences that bind to a target biomarker. This can be accomplished using The Systematic Evolution of Ligands by EXponential enrichment (SELEX) protocol as described previously in U.S. Pat. No. 5,712,375, performed in the presence of either a pure target biomarker or with the endogenous target biomarker in an analyte, bodily fluid, or other sample suitable for the SELEX process. After the SELEX process, the salimer-biomarker complex is treated with nucleases to digest any unbound portion of the salimer. The nuclease truncation of the salimer, while bound to its target biomarker, leaves only the core binding sequence, as it is protected by the interaction with its target biomarker. This identifies the minimal sequence required for interaction, which is the basis for the design of the primer, to guarantee competition with the target biomarker on binding this specific sequence, and to ensure that the affinity of the salimer for the biomarker is greater than the affinity of the salimer of the primer. Some embodiments of the present invention provide methods for identifying a salimer-biomarker binding sequence comprising contacting a salimer-biomarker complex with at least one nuclease under conditions amenable for oligonucleotide digestion; isolating the salimer-biomarker complex from the nuclease; dissociating the salimer from the biomarker; and identifying the salimer-biomarker binding sequence.

In some aspects of the present invention, the primer, salimer, or both are resistant to nucleases. Nuclease resistant refers to exonuclease resistance, endonuclease resistance, or a combination thereof. Nuclease resistance may be due to use of the any of the non-naturally occurring or modified nucleotides described above or achieved by introduction of additional modifications to the group described above. For example, primers and salimers having phosphorothioate bonds linking the nucleotides or having a 3′ phosphorylated end may be resistant to nuclease as well as primers and salimers containing modified nucleotides such as 2′-fluorobases.

A salimer can be modified to either increase or decrease its affinity for a primer. In some embodiments, a salimer can be modified to include a moiety that increases the salimer's affinity for the biomarker. In some aspects, the moiety can be an antibody, a lipid, a protein, a peptide, or a polypeptide. In some embodiments of the present invention, the salimer's affinity for a biomarker is greater than the salimer's affinity for the primer with which it hybridizes.

In some embodiments, a salimer is between about 5 to about 50 nucleotides in length. In some embodiments of the present invention, a salimer can also be about 60, 70, 80, 90, or even 100 nucleotides in length. In some embodiments, the primer is between about 10-20 nucleotides in length, between about 20-50 nucleotides in length, or even between about 50-100 nucleotides in length.

The extent of hybridization between the salimer and the primer depends, in part, on the sequences similarity between the oligonucleotide. The more complementary a primer's sequence is to a salimer's sequence, the higher the affinity constant and the greater the energy required to melt the hybridized oligonucleotides. Therefore, salimers and primers can be designed to achieve a desired affinity constant. For example, salimers and primers can be designed to have about 10%, about 20%, about 30%, about 40%, about 50%, about 60%, about 70%, about 80%, about 90%, or even about 100% sequence identity. In some aspects of the present invention, the primer and the salimer comprise nucleotide sequences that are at least 25% complimentary. In some aspects of the present invention, the primer and the salimer comprise nucleotide sequences that are at least 50% complimentary. In some aspects of the present invention, the primer and the salimer comprise nucleotide sequences that are at least 75% complimentary. In some aspects of the present invention, the primer and the salimer comprise nucleotide sequences that are 100% complimentary.

In some aspects of the present invention, the primer will be designed to hybridize to a single salimer, while in other aspects the primer will be designed to hybridize to more than one salimer. The multiple-salimer embodiment may allow for the simultaneous detection of more than one biomarker in an analyte. Similarly, the primer may be designed to hybridize to only one salimer having a particular sequence, while in other embodiments the primer may be designed such that the primer can hybridize to salimers having different sequences.

“Electrode” as used herein refers to a conductor built on a substrate. As direct contact of the surface of the electrode with a primer may damage the electrode, a primer can be considered “disposed on the surface of an electrode” when the primer is capable of electrical communication with the electrode. In some embodiments, the surface of the electrode is biofunctionalized with e.g. (3-glycidoxypropyl)trimethoxysilane (GOPS) or (3-aminopropyl)triethoxysilane (APTES) or (3-Mercaptopropyl)trimethoxysilane (MPTMS) or other equivalent materials, such that the biofunctionalized surface is suitable for contacting at least one primer and allowing electrical communication between the primer and the electrode. In some aspects, the biofunctionalized surface comprises a linker layer configured to contact and immobilize one end of the primer. In other aspects, the primer can be reversibly immobilized on the biofunctionalized surface of the electrode. The invention also contemplates modifying one end of the primer to allow contact with the surface of the electrode, the modification being such that the possibility of damage to the electrode is minimized or eliminated. An electrode can be either metallic or nonmetallic. Replacement of primers, should the primers become degraded or if a different primer is needed to optimize hybridization to a salimer, can be accomplished with chemical manipulation of the linker layer. The invention calls for at least one electrode, and in some aspects the at least one electrode comprises a multi-electrode array.

Analytes can be obtained from many sources including, but not limited to, bodily fluids, cells, swabs, hair, and biopsies. Some aspects provide for analytes obtained from saliva, blood, urine, tears, sweat, nasal, genitals or any other body fluid. An analyte, regardless of source, will have components other than the biomarker or biomarkers to be assayed. Thus, another embodiment of the present invention provides for removal of non-biomarker components from the analyte, wherein the removal is accomplished by treating the sample with at least one antibody that bind at least one analyte component. In some aspects, the at least one antibody is a monoclonal antibody specific for a particular analyte component, or antigen, and is used to bind to the component prior to exposing the analyte to the reaction chamber. The antibody-antigen complex can be removed from the analyte, for example, by centrifugation or filtration. In some aspects of the present invention, the at least one antibody is a polyclonal antibody.

In some aspects, the analyte component to be removed includes one or more enzymes. Enzymes in a saliva sample that can disrupt the detection of biomarkers include, but are not limited to amylases, lysozymes, lipases, proline-rich proteins, histatins, cystatins, statherin, or peroxidases, or any combination thereof. The affinity of these enzymes for the salimers or primers can compromise the detection of biomarkers and any resulting false positive or false negative results can have negative health consequences. The presence of other enzymes, such as nucleases, can also have deleterious effects on the detection of biomarkers as the nucleases may degrade the primer, the salimer, or both. As proteinaceous enzymes are susceptible to degradation, one aspect of the invention provides deproteinizing the analyte. For example, metaphosphoric acid is used to deproteinize the analyte in some embodiments. Other deproteinizing agents are contemplated in the present invention.

Components of an analyte may, under certain conditions, exhibit nonspecific binding to the primer or salimer. Such nonspecific binding may inhibit the binding of a salimer to a target biomarker or otherwise inhibit the competition reaction. Thus, one aspect of the present invention provides adjusting a temperature of the analyte competition reaction to reduce non-specific binding of the salimer, the primer, or any component of the analyte, or a combination thereof. In some aspects, changing the temperature at which the assay is performed provides the kinetic energy necessary to overcome nonspecific binding but does not alter the specific binding of the salimer to the primer. Other methods of reducing non-specific binding are contemplated in the present invention including, but not limited to, adjusting ionic strength of the competition reaction, or trapping sample components in the analyte, or any combination of the methods provided herein.

In some instances, an excess of salimers may be used for hybridization to fully saturate the primers or at least to achieve sufficient number of primer-salimer complexes. In some embodiments, at least one wash step may be employed, such as dispensing a liquid buffer solution to remove any unbound salimer prior to exposing the double-stranded hybrid to the analyte. Unbound salimer can potentially bind to the biomarker of interest, preventing a bound salimer from disassociating from a primer, which leads to a loss of signal and an inaccurate estimate of the amount of target biomarker in an analyte.

“Target biomarker,” as used herein, refers to the molecule or combination of molecules in an analyte that the methods of the present invention are designed to detect. An analyte may be comprised of a plurality of biomarkers, but only target biomarkers are detected in the methods. In some aspects of the present invention, the target biomarker comprises a metabolite, nutrient, toxin, drug ingredient, microorganism, polypeptide, lipid, sugar, oligonucleotide, ion, organic molecule, or inorganic molecule, or their derivatives or combinations thereof.

A “competition reaction” as described herein refers to a binding assay, in which two compounds compete to bind a third compound present in the reaction. In the present invention, the primer and the biomarker compete to bind with the salimer. In the presence of a biomarker, a salimer may dissociate from the primer, if the affinity of the salimer for the biomarker is greater than the affinity of the salimer of the primer. In the presence of a sample, the salimer-primer hybrid will only disassociate if a biomarker to which the salimer preferentially binds is present. In some embodiments, the affinity constant of a salimer and the salimer's biomarker will be greater than the affinity constant of the primer and the salimer. In most preferred embodiments, the affinity constant of the salimer and the biomarker will be significantly greater than the affinity constant of the salimer and the primer, such that in a mixture of primer, salimer, and biomarker, the salimer will bind, hybridize, or otherwise interact with the available biomarker. Some aspects of the present invention, in which the affinity of the salimer for biomarker is not sufficiently greater than affinity of the primer for the salimer, include adjusting the temperature of the competition reaction to partially melt the salimer-primer hybrid.

Another embodiment provides adjusting the temperature of the competition reaction between the primer and the biomarker can also alleviate non-specific binding or altered affinities due to reaction conditions. Some aspects of the present invention include adjusting an ionic strength of the competition reaction to reduce non-specific binding of the salimer, the primer, or any component of the analyte, or a combination thereof. For example, raising or lowering the pH of the competition reaction may provide the ionic environment necessary to ensure salimer-primer hybridization. Similarly, the salt concentration of the competition reaction may be adjusted to reduce the likelihood of a non-specific interaction involving the primer, the salimer, or both.

In some aspects of the invention, a population of primers and salimers will be designed to detect the presence of a single biomarker, such that in the presence of a large concentration of the biomarker, the majority or all of the salimers will dehybridize from the primer and interact with the biomarker, which results in a majority or all of the primers being single-stranded. Conversely, in the presence of a small concentration of the biomarker, the majority of primers and salimers will remain hybridized. In comparison, a greater total electronic signal will be produced for the sample containing a high concentration of biomarkers compared to a sample containing a small concentration of biomarkers. Therefore, one aspect of the present invention provides measuring the difference between the electronic signal detected from the double-stranded hybrid and the electronic signal detected from the single-stranded primer, wherein a greater single-stranded primer electronic signal compared to the double-stranded hybrid electronic signal indicates the presence of a biomarker.

The molecular principle of quantification in this invention is that the degree of removal of salimers from the salimer-primer hybrids is proportional to the concentration of the target biomarker in the tested analyte. For this purpose, at least a first electronic signal is detected from the electrode after primer-salimer hybridization and wash of the unbound salimers. Electronic signals may be continuously monitored in some aspects of the present invention. Addition of the analyte results in a competition for binding of the salimer, which leads to dehybridization of salimers from the primer, followed by wash of the unbound salimer-biomarker complex. At least a second electronic signal is detected from the electrode. The electronic signal from a single-stranded primer will be different than an electronic signal from a double-stranded hybrid. Electronic signal include, but not limited to, voltammetric, amperometric/coulometric, potentiometric, conductometric, and impedimetric signals. In some aspects, a different second electric signal relative to the first electrical signal indicates the presence of the target biomarker in the analyte. The electrical principle of quantification in this invention is that the difference between the electrical signals is proportional to the concentration of the target biomarker in the tested analyte. In some embodiments, the electronic signal will be quantified such that comparison to control values for a particular biomarker can be made. A known amount of a control compound may be included in an analyte to provide a reference signal to compare to a biomarker signal.

Electronic signals, such as those produced in the present invention, can be detected in a variety of ways. The electronic signal can be a visual or audio alarm. In other embodiments, the electronic signal can be an electronically communicable message. In some aspects of the present invention, a mobile device is used to detect the signal. The mobile device is used to manage data collected from the device. The mobile device is characterized as a cellular communications device.

The electronic signal produced by a single-stranded primer, in some embodiments, may be augmented to enhance the difference between the electronic signal produced by the salimer-primer hybrid and the single-stranded primer, thus improving the resolution of the results. In some embodiments, the single-stranded primer forms a secondary structure. During hybridization, the primer will lose its secondary structure as it anneals to the at least partially complementary to the sequence of the salimer. Upon dissociation of the salimer and primer hybrid, the single stranded primer, in some embodiments, will reform a secondary structure. This secondary structure enhances the electrical signal generated by the single stranded primer. In other embodiments, the primer will not form a secondary structure. In some embodiments, a signal enhancer molecule is attached to the primer. Signal enhancers can be nanoparticles, chemical moieties, or other compounds. For example, in one aspect, the signal enhancer comprises an electron transfer moiety, which can facilitate electron relocation from one or more of its atoms to the electrode when in close proximity. In some aspects, the electron transfer moiety comprises a transition metal complex. As used herein, “metal transition complex” refers to a central metal ion bound by an array of surrounding ions or molecules. Also known as a coordination complex, the metal ion of a metal transition complex is a transition metal ion such as iron or ruthenium. In some aspects of the present invention, the metal transition complex comprises a metallocene. In some embodiments, the metallocene comprises a ruthenium complex.

In one aspect of the present invention, a fluorophore is attached to the primer and the salimer is attached to a dark quencher. A “dark quencher” as used herein refers to a substance that absorbs excitation energy from a fluorophore. When the primer and salimer are hybridized, the dark quencher minimizes or prevents a fluorescence signal from the fluorophore. When the salimer is complexed with a biomarker, it will not be in proximity to the fluorophore attached to the primer, and the fluorophore will emit a visibly detectable signal. The fluorescence can be detected and measured using a fluorescence detector. In some qualitative embodiments, the fluorescence emitted is visually detected. Some aspects of the present invention provide methods for detecting the presence or absence of a biomarker in an analyte comprising hybridizing a fluorophore-labeled primer to a dark quencher-labeled salimer to form a double-stranded primer-salimer hybrid; detecting a first fluorescence signal from; exposing the analyte to the double-stranded hybrid to initiate a competition reaction between the primer and the target biomarker for complexing with the salimer, wherein the salimer dissociates from the primer in the presence of the target biomarker and forms a complex with the target biomarker; detecting a second fluorescent signal; and comparing the fluorescent signals to ascertain the presence or absence of the target biomarker.

Some embodiments of the present invention provide for serial reactions. Because the primer is disposed on the electrode, it is suitable for multiple reactions, while other components are removable. Salimers may need to be replaced as they may be degraded or otherwise become less than optimal during interaction with components of the analyte. In some embodiments, therefore, analyte and salimers are removed after the competition reaction but before the second electrical signal is detected. In some aspects, after the second signal is detected, salimers that remain hybridized to the primer are removed, leaving just the primers disposed on the surface of the electrode.

In one aspect of the present invention, the primer is capable of hybridizing with different salimers. For example, in a biomarker competition reaction the primer hybridizes to a salimer that preferentially binds to a biomarker associated with diabetes. In a subsequent biomarker detection reaction, a salimer that preferentially binds to a cancer-associated biomarker is hybridized to the primer. Thus, the presence or absence of many biomarkers can be assessed in sequential detection reactions.

Other embodiments of the present invention include employing multiple primers, or populations of primers in an array of electrodes, each primer having the capacity to recognize and hybridize to one specific salimer of a mix of various salimers dispensed together, each of which preferentially binds to a different biomarker. This allows for parallel reactions and the simultaneous detection of multiple biomarkers, which increases efficiency by eliminating the need for multiple sequential reactions and sample collection. Similar embodiments include physically segregating more than one primer that hybridize with salimers that preferentially bind with the same biomarker. Such embodiments allow for parallel reactions probing for the same biomarker, providing greater certainty in the determination of the presence or absence of the biomarker.

After completion of the competition reaction, at least one wash step may be employed, such as dispensing a liquid buffer solution to remove the unbound salimer-biomarker complex, and other materials originate in the analyte, prior to measuring the second signal of the electrode.

After completion of the measurement the second signal of the electrode, the residual hybridized salimers can be removed from the primers to enable the surface of the electrode to be reused. Washing the electrode with a solution may efficiently remove analytes, salimers, contaminants, and other compounds on the electrode surface or in a reaction chamber. For this reason, some embodiments of the present invention include dispensing buffer on the surface of the electrode to dissociate salimers from their complementary primers and a buffer wash to remove unbound salimers. Other aspects include heating the surface of the electrode to dissociate salimers from their complementary primers and a buffer wash to remove unbound salimers.

To improve the signals from the electrode, a buffer change or a wash step can be employed. This will remove excess reaction components (e.g., unhybridized salimers or analyte components). Used buffers, wash reagents, and other used materials can be polymerized in a separate chamber to create a gel, for example by using superabsorbent polymers (SAPs), with or without UV radiation to drive the polymerization and cross-linking reactions.

### EXAMPLES

To assess the presence, progression, or absence of cardiovascular disease in a subject, samples are obtained from the subject to be tested for the presence or absence of cardiac troponin T (cTnT), a biomarker of cardiovascular disease and possible myocardial infarction (MI). A volume of 100 μL of buffer is dispensed three times on the surface of the electrode. Primers are disposed on the surface of the electrode. After the third wash, a pre-mix of salimers is dispensed onto the surface of the electrode. Each salimer has at least a 10-fold greater affinity for cTnT than for the primers disposed on the electrode surface. The salimers also have concentrations less than or equal to 100 ng/ml. Hybridization is carried out for 30 seconds at 37° C. (enhancement of hybridization can be facilitated through manipulation of surface charges of the electrode). Three additional washes are applied to the electrode to remove excess and unbound salimers. After the third wash, an electrical measurement is taken to establish a baseline. This measurement reflects the number of hybridized primer-salimers on the electrode.

50 μL of sample, mixed with 50 μL of buffer is dispensed onto the surface of the electrode and incubated with the hybridized primer-salimer molecules for 120 seconds at 37° C. During this period, the salimers disassociate from the primer and interacts with biomarkers present in the sample. Other contents of the sample and dissociated molecules are removed by washing the electrode twice with 100 μL of buffer. An electrical measurement is taken, and the difference from baseline is determined. Finally, 100 μL of buffer is dispensed twice onto the surface of the electrode at 50° C. to allow dehybridization of primer-salimer pairs still bound to the surface, to enable the reuse of the electrode for an additional test. Those skilled in the art will appreciate that numerous changes and modifications can be made to the preferred embodiments and examples of the invention and that such changes and modifications can be made without departing from the spirit of the invention. It is, therefore, intended that the appended claims cover all such equivalent variations as fall within the true spirit and scope of the invention.

What is claimed:

1. A method of determining the presence or absence of a target biomarker in an analyte comprising:
hybridizing a salimer to a primer immobilized on a surface of an electrode, the electrode capable of being electrically energized, at a temperature suitable to form a double-stranded hybrid;
detecting a first electrical signal from the electrode with the double-stranded hybrid disposed on the surface of the electrode;
exposing the analyte to the double-stranded hybrid to initiate a competition reaction between the primer and the target biomarker for complexing with the salimer, wherein the salimer dissociates from the primer in the presence of the target biomarker and forms a complex with the target biomarker;
detecting a second electrical signal from the electrode; and
comparing the first and second electrical signals to ascertain the presence or absence of the target biomarker.

2. The method of claim 1, wherein a difference in the second electric signal relative to the first electrical signal indicates the presence of the target biomarker in the analyte.

3. (canceled)

4. The method of claim 2 further comprising measuring a difference between the first and second electrical signals, wherein the difference is proportional to the amount of the target biomarker in the analyte.

5. The method of claim 1, wherein the analyte comprises a known amount of a control compound.

6. The method of claim 1, wherein the target biomarker comprises a metabolite, nutrient, toxin, drug ingredient, microorganism, polypeptide, lipid, sugar, oligonucleotide, ion, organic molecule, or inorganic molecule, or their derivatives or combinations thereof

7. The method of claim 1, wherein the primer comprises a single-stranded oligonucleotide of DNA, RNA, or modified nucleotides, or combinations thereof, and the primer is between about 10-20 nucleotides in length, between about 20-50 nucleotides in length, or between about 50-100 nucleotides in length.

8-11. (canceled)

12. The method of claim 1, wherein the salimer comprises DNA, RNA, or modified nucleotides, or combinations thereof

13. The method of claim 12, wherein the salimer is resistant to nucleases.

14. The method of claim 12, wherein the salimer is between 10-20 nucleotides in length, between about 20-50 nucleotides in length, or between about 50-100 nucleotides in length.

15-16. (canceled)

17. The method of claim 1, wherein the salimer has greater affinity for the target biomarker than for the primer.

18. The method of claim 7, wherein the primer and the salimer comprise nucleotide sequences that are between 25% complimentary to 100% complementary.

19-21. (canceled)

22. The method of claim 1, wherein the primer is single-stranded and forms a secondary structure.

23. The method of claim 1, further comprising adjusting the temperature suitable to form a double-stranded hybrid to facilitate removal of any unbound salimer prior to exposing the double-stranded hybrid to the analyte.

24-26. (canceled)

27. The method of claim 1 further comprising treating the analyte with at least one antibody that binds at least one analyte component comprising amylases, lysozymes, lipases, proline rich proteins, histatins, cystatins, statherin, or peroxidases, or combinations thereof.

28-29. (canceled)

30. The method of claim 1 further comprising deproteinizing the analyte.

31-33. (canceled)

34. The method of claim 1, further comprising a signal enhancer molecule attached to the primer, wherein the signal enhancer molecule comprises an electron transfer moiety comprising a transition metal complex.

35-41. (canceled)

42. The method of claim 1, wherein the analyte is obtained from saliva, blood, urine, tears, sweat, nasal, genital, or any other body fluid.

43-45. (canceled)

46. A method of determining the presence or absence of a biomarker in an analyte comprising:
in a reaction chamber, hybridizing at least one single-stranded salimer to at least one single-stranded primer to form at least one double-stranded hybrid, wherein the reaction chamber comprises an interior, an exterior, an inlet connecting the exterior of the chamber and the interior of the chamber, and at least one electrode capable of being electrically energized, wherein the at least one primer is disposed on a surface of the at least one electrode, and wherein the at least one salimer has a greater affinity for a target biomarker than for the at least one primer;
detecting a first electrical signal from the electrode with the double-stranded hybrid disposed on the surface of the electrode;
delivering the analyte to the interior of the chamber, wherein in the presence of the target biomarker the at least one salimer preferentially interacts with the target biomarker and dissociates from the at least one primer to form a salimer-biomarker complex;
detecting a second electrical signal from the at least one electrode; and comparing the first electrical signal to the second electrical signal, wherein a difference between the first signal and the second signal indicates the presence of a biomarker.

47. The method of claim 46, wherein the at least one electrode comprises a multi-electrode array.

48-59. (canceled)

60. A method of detecting the presence or absence of a biomarker in an analyte comprising:
hybridizing a fluorophore-labeled primer to a dark quencher-labeled salimer to form a double-stranded primer-salimer hybrid; detecting a first fluorescence signal;
exposing the analyte to the double-stranded hybrid to initiate a competition reaction between the primer and the target biomarker for complexing with the salimer, wherein the salimer dissociates from the primer in the presence of the target biomarker and forms a complex with the target biomarker;
detecting a second fluorescent signal; and
comparing the fluorescent signals to ascertain the presence or absence of the target biomarker.

61. (canceled)
